(19) United States
(12) Patent Application Publication
(10) Pub. No.: US 2020/0337847 A1
(43) Pub. Date: Oct. 29, 2020

(54) GRAFT FOR REPAIRING ARTICULAR CARTILAGE DEFECTS AND METHOD FOR THE SAME

(71) Applicant: Shanghai Sixth People's Hospital, Shanghai (CN)

(72) Inventors: Changqing Zhang, Shanghai (CN); Dajiang Du, Shanghai (CN)

(73) Assignee: Shanghai Sixth People's Hospital, Shanghai (CN)

(21) Appl. No.: 16/530,123

(22) Filed: Aug. 2, 2019

(30) Foreign Application Priority Data
Apr. 23, 2019 (CN) 201910330808.9

(51) Int. Cl. A61F 2/30; A61L 27/38; A61L 27/52; A61L 27/36

(52) U.S. Cl. CPC A61F 2/30756; A61L 27/3817; A61L 27/24; A61L 27/3654; A61L 2430/06; A61L 27/52

(57) ABSTRACT

The invention relates to a graft for repairing articular cartilage defects, comprising at least one of an autologous costal cartilage or an allogeneic costal cartilage, wherein the graft can be a whole piece of costal cartilage or a cartilage particle-bound hydrogel graft. The invention further relates to the use of the graft and a method for repairing articular cartilage defects. In the present invention, it has a small secondary injury by using costal cartilage implantation. In addition, it can be a minimally invasive operation, avoiding the risk of complications such as prosthesis loosening and infection due to the artificial joint replacement. Because the amount of costal cartilage is sufficient, it can meet the needs of multiple cartilage reconstruction and revision surgeries. Individualized reconstruction of injured articular cartilage surface can be achieved, which provides a safer, more operable repair method for patients diagnosed with articular cartilage defects.

### CROSS-REFERENCE TO RELATED APPLICATIONS

The present application claims priority to and the benefit of Chinese Patent Application No. CN 201910330808.9 filed on Apr. 23, 2019, the entire contents of which is incorporated herein by reference.

### BACKGROUND OF THE INVENTION

### 1. Field of the Invention

The invention relates to the technical field of medical and biomedical engineering, and more particularly, to a graft for repairing articular cartilage defects and a method for the same.

### 2. Description of the Related Art

Articular cartilage is a hyaline cartilage and mainly consists of cartilage cells, cartilage matrix and type-II collagen. It has complex biomechanical characteristics and high durability. However, it has a poor self-healing ability, thus defects due to factors such as natural degeneration and injury may result in irreversible damage in terms of structure and functions.

Since the artificial joint has a limited lifespan, for young adults, when hip and knee joint cartilages are damaged, if an artificial joint replacement is performed, then multiple revision surgeries may be required. Therefore, when trauma, necrosis, infection and other factors lead to the degeneration and damage of cartilage in the articular surface, artificial joint replacement should be avoided, and actively attempt to carry out treatment without removing original joints, mainly aiming to repair and reconstruct cartilage. However, there are no nerves or blood vessels in adult cartilage tissues, so those cartilage tissues are not regenerative and self-healing. Now commonly used methods to repair and reconstruct cartilage comprise microfracture, allogeneic osteochondral transplantation (including bone components), non-weight-bearing autologous osteochondral transplantation, etc. However, the above-mentioned methods include the following disadvantages: graft cartilage degeneration, rejection, morphological fit difficulty, and graft site complications, therefore, they are not widely recognized by those skilled in the art. In addition, there is no known method for effectively repairing and reconstructing cartilage on a femoral head.

In recent years, with the progress of cytohistology, autologous chondrocyte implantation (ACI) and matrix-induced autologous chondrocyte implantation (MACI) have been applied to the treatment of cartilage defects in a human knee joint. However, although the repairable area is large, the clinical efficacy is poor when the cartilage defects have an impact on the subchondral bone due to the fact that the early mechanical strength of the transplanted cells and the existing scaffold materials is poor. There is a lack of histological evidence that autologous chondrocytes can maintain cartilage phenotype and avoid dedifferentiation after culture and transplantation, and the autologous chondrocytes form hyaline cartilage-like tissues instead of true hyaline cartilage. Furthermore, the first three generations of chondrocyte implantation techniques for clinical applications are a two-stage surgery, which is expensive, technically complex, and demanding on facilities.

The costal cartilage is a hyaline cartilage, and its tissue morphology is close to that of articular cartilage, so it can be a reliable source of autologous cartilage for reconstruction of an articular cartilage surface. At present, costal cartilage implantation has been routinely applied to the reconstruction of the external ear and trachea. Rib-cartilage joints are used for the repair and reconstruction of cartilage defects on interphalangeal joints, metacarpophalangeal joints, temporomandibular joint, elbow joints and wrist joints. However, one rib has only one osteochondral joint, in this case, mosaic repairing a large area of osteochondral defects by using a plurality of rib-cartilage joints may lead to secondary injury of a donor site of the patient. Therefore, it is difficult to apply such a method to the repair and reconstruction of large-scale cartilage defects of large joints, and its relevant document report is not yet available.

Therefore, for patients with articular cartilage defects, there is an urgent need of a repair graft that is safer, easier to operate, and cost-effective.

### SUMMARY OF THE INVENTION

In order to overcome the problems in the prior art, embodiments of the present invention provide a graft for repairing articular cartilage defects and methods for the same. An autologous costal cartilage and/or an allogeneic costal cartilage are used to prepare the graft, so as to repair the articular cartilage defects.

To this end, the detailed technical solution provided in the present invention is as follows:

A first aspect of the present invention provides a graft for repairing articular cartilage defects, wherein the graft comprises autologous hyaline cartilage and/or allogeneic hyaline cartilage, wherein the hyaline cartilage is selected from the group consisting of at least one of articular cartilage and costal cartilage.

In order to optimize the technical solution of the graft, embodiments of the present invention comprise the following technical measures:

The fourth generation of chondrocyte implantation technology in a single-stage surgery has become a research hotspot in the art. Use of allogeneic articular cartilage granules combined with hydrogel has been clinically applied to the first-stage repair of knee joint osteochondral defects, and has achieved good clinical effects, but its source is quite limited and it is complicated to prepare. Allogeneic costal cartilage has many advantages over articular cartilage, such as a large reserve, a single source of tissues and an easy processing method. Therefore, allogeneic costal cartilage is better. in addition, research has shown that costal cartilage, with a bone bed, may form a reliable biological binding interface again after being implanted into cartilage defects and no obvious change has been found in hyaline cartilage tissue morphology. Therefore, it is theoretically feasible to repair a large area of articular cartilage defect by implanting a whole piece of costal cartilage or by mosaic implanting multiple parts of costal cartilage that is cut.

Therefore, a preferred technical solution of the present invention is a graft comprising at least one of an autologous costal cartilage or an allogeneic costal cartilage.

Preferably, the costal cartilage does not have an osteochondral joint.

Preferably, when autologous costal cartilage is used, the autologous costal cartilage is selected from the fifth to seventh ribs, more preferably the sixth costal cartilage. When allogeneic costal cartilage is used, the cartilage is any horizontal costal cartilage.

Preferably, the costal cartilage can be directly implanted to a cartilage defect to be repaired.

Preferably, the costal cartilage is a whole piece of costal cartilage or granular cartilages.

Preferably, the granular cartilages have a size between 0.1 mm to 1.5 mm.

Preferably, the graft further comprises a hydrogel system, and the granular cartilages account for 20%-90%, more preferably 30%-85%, based on the total weight of the hydrogel system.

Preferably; the hydrogel system is selected from the group consisting of autologous fibrin, autologous platelet-rich plasma (PRP), synthetic collagen, gelatin, chitosan, and polymer hydrogel. More preferably, the hydrogel system is a phototriggered-imine-crosslinking (PIC) hydrogel system, which may be constructed by hyaluronic acid (HA) and gelatin (GL). The preparation of a hyaluronic acid molecule HA-NB (hyaluronic acid-nitrobenzyl alcohol) via photochemical reaction is as follows:

Synthetic route of nitrobenzyl alcohol (NB)

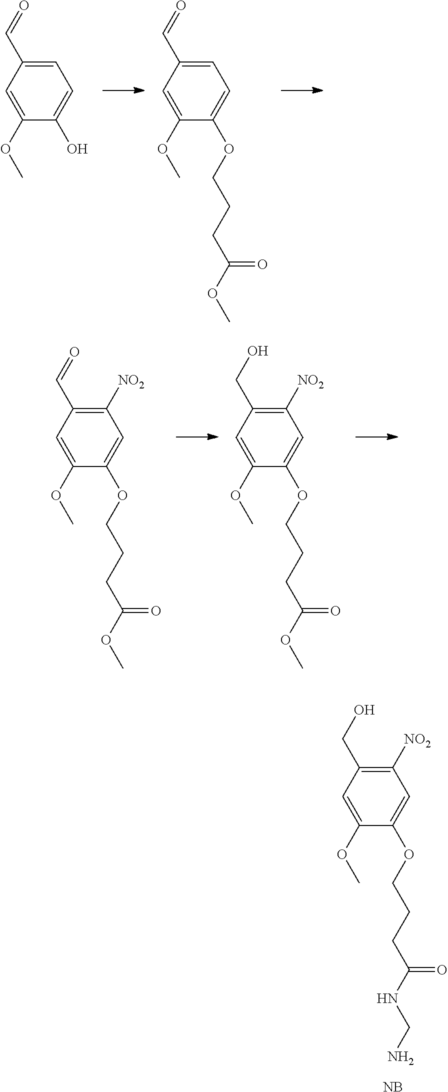

Synthetic route of HA-NB

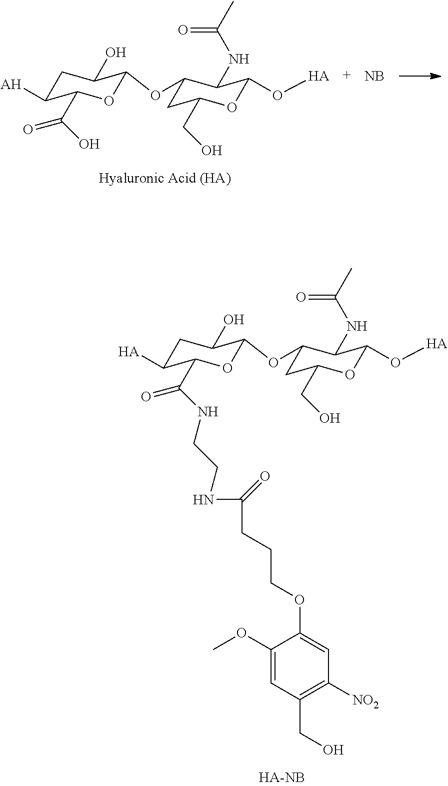

It should he appreciated that the PIC hydrogel system suitable for use in conjunction with costal cartilage can be prepared by using other suitable methods in the art. In addition, it should also be understood that a hydrogel system suitable for use with the costal cartilage can be obtained or prepared by using conventional methods in the art.

Preferably, the allogeneic costal cartilage is an allogeneic costal cartilage of a young donor, particularly a costal cartilage of a juvenile donor, wherein the juvenile donor is 3-18 years old; when the same allogeneic costal cartilage is used, a whole piece of allogeneic costal cartilage can be implanted, or granular cartilages may be implanted.

A second aspect of the present invention provides a method for constructing a graft for repairing articular cartilage defects. The graft includes costal cartilage particles and a hydrogel system. The method comprises the following steps: collecting at least one of an autologous costal cartilage or an allogeneic costal cartilage, cutting the same into particles having a size of 0.1 mm to 1.5 mm, and mixing cartilage particles with the hydrogel system, so as to form the graft, wherein the granular cartilages account for 30%-85% of the total weight of the system.

Preferably, in methods for constructing a graft for repairing articular cartilage defects, the cartilage particles can be pre-stored in a cartilage graft preservation solution (serum-free medium and a pH buffer). The preservation solution is preferably removed before using the cartilage particles, then the cartilage particles are mixed with the hydrogel system so as to form the graft.

A third aspect of the present invention provides a use of autologous costal cartilage or allogeneic costal cartilage in the preparation of a graft for repairing articular cartilage defects.

Preferably, an articular cartilage defect refers to a large area of cartilage injury caused by trauma, degeneration and other factors, wherein the cartilage injury comprises cartilage defect on a femoral head, cartilage denudation caused by avascular necrosis of the femoral head, or cartilage defect on a knee joint, such as exfoliative osteochondritis, osteoarthrosis, atic cartilage damage, etc.

A fourth aspect of the present invention provides a method for repairing articular cartilage defects, which applies any one of the grafts described above to a cartilage defect site to be repaired.

Preferably, in a first method for repairing articular cartilage defects, a graft is an autologous costal cartilage or an allogeneic costal cartilage, and the articular cartilage defect is a cartilage defect on a femoral head or a large area of osteochondral defect on a knee joint and the method comprises the following steps:

(1) in an injured articular cartilage surface, clearing necrotic and degenerative cartilages and subchondral bone tissues, and preparing a viable bone bed for implantation of the costal cartilage;

(2) cutting out a corresponding length of the costal cartilage depending on a size of the cartilage defect for collecting the autologous costal cartilage or the allogeneic costal cartilage; and (3) implanting the autologous costal cartilage or the allogeneic costal cartilage into the bone bed formed by the removal of the cartilage defect, securing the autologous costal cartilage or the allogeneic costal cartilage in the bone bed (if necessary, by means of additional absorbable screws or countersunk screws) and cutting the same into desired shapes, so as to match the cartilage surface with a surrounding cartilage surface and articular surface morphology.

Preferably, in step (3), the specific steps for implanting the autologous costal cartilage is selected from:

1) in the case of larger defect area, slitting the costal cartilage longitudinally or splicing the costal cartilage in segments for implantation, and fixing the costal cartilage with absorbable screws;

2) perforating the injured cartilage surface, and press fitting and embedding the repaired costal cartilage into the hole vertically for implantation, cutting the cartilage surface subjected to repair and implantation, to make it smoother and higher than its surrounding area by 1 mm to 2 mm; or

3) if the depth of the cartilage defect site is deep, taking the iliac bone for bone grafting; repairing the bone defect with the iliac bone, grafting the costal cartilage on the surface and fixing in place by press fitting or using additional absorbable screws, and cutting the same into desired shapes, so as to match the cartilage surface with a surrounding cartilage surface and articular surface morphology.

Preferably, the first method for repairing articular cartilage defects by using autologous costal cartilage comprises the following steps:

1. exposing the cartilage defect and debriding the cartilage defect: after anesthesia, making an appropriate incision on the surface of the cartilage defect site to expose the articular cartilage surface, and clearing the necrotic degenerative cartilage and subchondral bone tissues until fresh bleeding occurs in the bone bed;

2. collecting the autologous costal cartilage: it is preferably, although not essential, that collecting the costal cartilage on the right side since it is farther away from the heart; due to the fact that the costal cartilage is longer and it has a larger diameter, the fifth to seventh ribs are preferably selected, and the sixth rib is often the optimum choice; an incision is made on the skin along a long axis of the sixth rib, and taking care to avoid damaging intercostal vessels and nerves during this process; cutting out a costal cartilage of a corresponding length depending on the size of the cartilage defect; and after confirming that pleura is intact without any injury, closing the incision on the chest;

3. implanting the autologous costal cartilage: after repairing the costal cartilage collected in step 2, the costal cartilage is implanted into the bone bed prepared in step 1 and is fixed in place, then it is cut into desired shape, so as to match the cartilage surface with a surrounding cartilage surface and articular surface morphology; exercising joints in all directions to confirm that the costal cartilage is stable, and that no impingement occurs and abnormal contact is not found in the joints, then rinsing, suturing and closing the incision; and

4. postoperative rehabilitation: after the surgery, a patient is asked to exercise the joints passively in full range; on the second day after the surgery, the patient can walk by the aid of crutches; after two weeks from the surgery, the patient begins to carry out full-range of exercises actively, and to train on the muscle strength step by step; and after three months from the surgery, full weight-bearing walking is regained gradually depending on the recovery status.

Preferably, the first method for repairing articular cartilage defects by using allogeneic costal cartilage comprises the steps which are substantially the same as those of the first method for repairing articular cartilage defects by using autologous costal cartilage. The only one difference is that in step 2, the collected costal cartilage is any horizontal costal cartilage from any suitable young donor.

Preferably, in a second method for repairing articular cartilage defects, the graft is autologous costal cartilage or allogeneic costal cartilage, and the articular cartilage defect is a knee joint osteochondral injury and the method comprises the following steps:

(A) collecting at least one of autologous costal cartilage or allogeneic costal cartilage, cutting the same into particles having a size of 0.1 mm to 1.5 mm, and mixing cartilage particles with a hydrogel system, so as to form the graft consisting of the costal cartilage particles and the hydrogel system, wherein the granular cartilages account for 30%-85% of the total weight of the system;

(B) clearing necrotic and degenerative cartilage and subchondral bone tissues from the knee joint osteochondral injury and preparing a bone bed to be processed; and

(C) filling in the graft to form an integrated seamless surface, and facilitating integration of new cartilages into surrounding cartilage tissues.

It will be understood that the above-mentioned costal cartilage may be replaced by any suitable hyaline cartilage, such as articular cartilage, etc.

By adopting the above-mentioned technical solutions, the present invention has the following technical effects when compared with the prior art:

1. in terms of secondary injury, compared with traditional autologous knee joint or femoral head non-weight-bearing cartilage implantation, the autologous costal cartilage implantation does not damage the normal articular surface, and the secondary injury is small, and such an implantation can be performed by a minimally invasive procedure, therefore, complications of knee pain and osteoarthrosis may be prevented;

2. in histology, both autologous costal cartilage and articular cartilage are hyaline cartilage; not only the original rib-cartilage joint can be directly used for repairing cartilage, free costal cartilage can also form a bone-cartilage biobinding interface with the bone bed after its implantation; free allogeneic costal cartilage without bone tissues are used for implantation, since cells are embedded in the cartilage matrix, having an immunological shielding effect. When compared with traditional allogeneic osteochondral implantation, free allogeneic costal cartilage without bone tissues implantation rarely result in immune rejection;

3. in terms of the amount of cartilage than can be collected, when compared with the limited knee joint or femoral head non-weight-bearing area, the costal cartilage available for collecting is adequate. By using mosaic forming technology, a rib can repair a large area of osteochondral defects if cut into multiple segments, so as to reconstruct the surface morphology of the large joint, so the technique for repairing the jointed cartilage by using the autologous costal cartilage implantation technique to save the joints can also be called “one rib for one joint” technique; in addition, since the amount of costal cartilage is sufficient, it can even meet the needs of multiple cartilage reconstruction and revision surgeries;

4. in terms of shape controllability, when compared with articular cartilage, costal cartilage with a certain thickness can be repaired and shaped simply by using a scalpel, so that the surface of the cartilage after reconstruction and repair can be matched with the original shape and the surrounding cartilage surface morphology; in addition, by using 3D printing technology, individualized reconstruction of damaged articular cartilage surface may even be achieved, these advantages are not available in other cartilage repair and reconstruction methods; and,

5. The present invention also utilizes a hydrogel system and granulated costal cartilages for implantation, which can “bond” the active hyaline cartilage particles to the defect site to induce local tissue repair. The autologous ribs or allogeneic costal cartilages of a young donor are used to prepare a graft, consisting of costal cartilage particles and the hydrogel system, for repairing articular cartilage defects, through which minimally invasive treatment of the allograft costal cartilage may be achieved.

### BRIEF DESCRIPTION OF THE DRAWINGS

The accompanying drawings, together with the specification, illustrate exemplary embodiments of the present disclosure, and, together with the description, serve to explain the principles of the present invention.

FIG. 1 is a schematic view showing steps of repairing a cartilage defect on a femoral head by implanting autologous costal cartilage according to an embodiment of the present invention;

FIG. 2 is a schematic view showing steps of repairing cartilage defect on a knee joint by implanting autologous costal cartilage according to an embodiment of the present invention;

FIG. 3 is a schematic view showing steps of repairing cartilage defect on a knee joint by implanting allogeneic costal cartilage particles according to an embodiment of the present invention; and

FIG. 4 is a schematic view showing steps of repairing and reconstructing cartilage defect on a femoral head by using costal cartilage graft in a clinical application according to an embodiment of the present invention.

### DETAILED DESCRIPTION

The present invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Hereinafter, certain exemplary embodiments according to the present disclosure will be described with reference to the accompanying drawings.

The present invention relates to a graft for repairing articular cartilage defects, wherein the graft comprises at least one of an autologous costal cartilage or an allogeneic costal cartilage. The invention also relates to a use of the graft and a method for repairing articular cartilage defects.

The specific embodiments of the present invention are further described below in conjunction with the drawings and embodiments. The following examples are only intended to more clearly illustrate the technical solutions of the present invention, and are not intended to limit the scope of the present invention.

### EXAMPLE 1

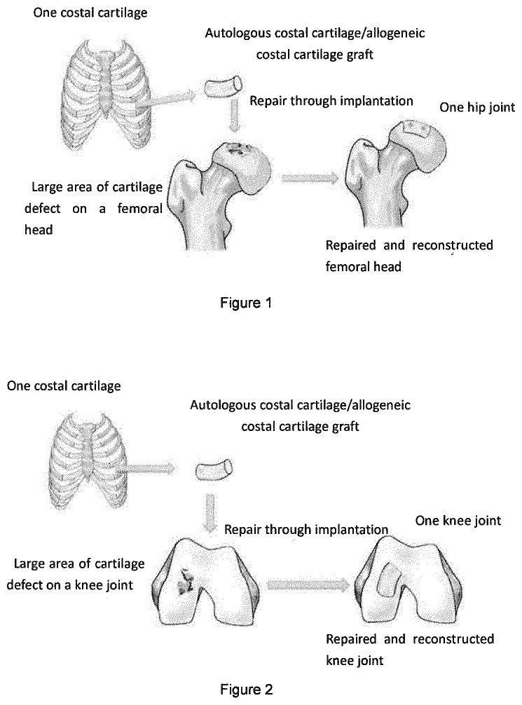

This example is a method for repairing cartilage defect on a femoral head by implanting autologous costal cartilage, and the method comprises the following steps, as shown in FIG. 1:

Under general anesthesia, the patient was placed in the supine position, incisions were made on skin, subcutaneous tissues, and fascia lata in sequence by using Smith-Peterson approach, but taking care to avoid damaging anterolateral cutaneous nerves, to fully expose the anterior joint capsule of the hip joint, and incisions were made in a “T”-shape. After fully loosening the hip joint, the femur is at adduction and extorsion, and the femoral head is exposed. The femoral head is thoroughly cleared of the degenerative necrotic bone and cartilage tissues The osteochondral detect site on the surface of the femoral head is identified. According to the severity of the bone defect, the iliac bone block on the same side in the same incision was collected to reconstruct the subchondral bone defect of the femoral head. While one group of doctors were performing the hip joint surgery, another groups of doctors stood to the right side to collect the costal cartilage, and made an incision on the skin along the long axis of the right 6th rib (moving window technique), wherein the incision was about 6 cm long, but taking care to avoid damaging the intercostal vessels and nerves. According to the size of the femoral head osteochondral detect, costal cartilage of about 5 to 6 cm long was cut out. After confirming that the pleura was intact without any injury, the rib periosteum was sutured, and the chest incision closed. The collected iliac bone block was repaired and embedded into the subchondral bone defect in the weight-bearing area of the femoral head The shape was trimmed; the whole piece of the costal cartilage was longitudinally split into two pieces with a scalpel, and then the two pieces of costal cartilage were implanted to the surface of the femoral head cartilage defect site. The costal cartilage and the iliac bone block were fixed with six absorbable screws and the surface of the costal cartilage was cut with the scalpel along the articular surface curvature of the femoral head to reshape the articular surface of the femoral head. Traction of the lower limbs, knee flexion, and inte a tation to reset the hip joint were performed, moving the hip joint in all directions, so as to confirm that the costal cartilage was stable after implantation. The costal cartilage was not in abnormal contact with the acetabular edge, such as impact. The range of passive motion of the hip joint was determined, and it was confirmed that the implanted cartilage was stable. The femoral head was good in shape through the observation of C-arm fluoroscopy. The joint capsule was sutured and rinsed with a large amount of normal saline layer by layer and all of the incisions were closed. After the surgery, the patient was asked to exercise a large range of motion of the joints passively in full range, and was prohibited from performing adduction and extorsion motions of the hip joint. On the second day after the surgery, the patient can walk by the aid of crutches. After two weeks from the surgery, the patient begins to carry out full-range of exercises actively, and to train on the muscle strength step by step; and after three months from the surgery, full weight-bearing walking is regained gradually.

### EXAMPLE 2

This example is a method for repairing a cartilage defect on a knee joint by implanting autologous costal cartilage comprising the following steps and as shown in FIG. 2:

The patient was placed in a supine position under general anesthesia. After sterilization and draping, exsanguination was performed on the affected limb to reduce bleeding. A medial parapatellar incision was made longitudinally in the left knee in superficial and deep fascia, and an incision was made on the media patellar retinaculum. After the patella was elevated laterally the cartilage lesion was identified in the medial femoral condyle. The degenerative cartilage and bone tissue of the lesion were thoroughly debrided until fresh bleeding occured in the bone bed. The ipsilateral iliac bone mass was collected to reconstruct the subchondral bone defect. At the same time as the knee surgery, another group of doctors stood on the right side of patient to collect the costal cartilage. A 4 cm incision along the long axis of the right 6th rib was made avoiding the intercostal vessels and nerves. The costal cartilage of a corresponding length was collected with tools such as a bone knife. After confirming that the pleura was intact without any injury, the rib periosteum was rinsed and sutured layer by layer and the chest incision closed. Sterile foil was pressed into the defect so that the outer shape of the foil fit snugly into the defect. The foil was removed from the defect. The costal cartilage was trimmed according to the size and shape of the foil and was grafted by press-fitting into the cartilage defect. After fixing the graft with absorbable screws, a scalpel was used to trim the surface of the costal cartilage along the articular contour of femoral condyle so as to reconstruct the curvature of the articular surface. After the patella was reduced, the knee was flexed and extended in full range to confirm the stability of the grafted costal cartilage and the smoothness of the motion. No friction or abnormal impingement was detected and the cartilage graft was also confirmed stable. The intraoperative C-arm radiography showed that the knee joint gap was balanced and alignment of the lower limb was good. After careful hemostasis, the incision was rinsed and closed in layers. Postoperatively, the patient began passive knee motion for the first day. Mobilization on crutches was allowed without weight-bearing on the second day, At 4 weeks postoperatively, the knee motion was from 0 ° to 120°. After 12 weeks, weight bearing was progressed gradually as tolerated.

### EXAMPLE 3

This example is a method for repairing a cartilage defect on a femoral head by implanting allogeneic costal cartilage comprising the following steps:

Collecting and storing the allogeneic cartilage: costal cartilage from a young donor was collected and placed in a preservation solution (serum-free medium, pH buffered), and the preservation solution was removed during use;

Under general anesthesia, the patient was placed in the supine position, incisions were made on skin, subcutaneous tissues, and fascia lata in sequence by using Smith-Peterson approach, but taking care to avoid damaging anterolateral femoral cutaneous nerves, to fully expose the anterior joint capsule of the hip joint, and incisions were made in a “T”-shape. After fully loosening the hip joint, the femur was at adduction and extorsion, and the femoral head was exposed. The femoral head was thoroughly cleared of degenerative necrotic bone and cartilage tissues. The osteochondral detect site on the surface of the femoral head was identified. According to the severity of the bone defect, the iliac bone block on the same side in the same incision to reconstruct the subchondral bone defect of the femoral head was collected. The iliac bone block was repaired and embedded into the cartilage subchondral bone defect in the weight-bearing area of the femoral head. The shape was trimmed. The allogeneic cartilage was collected and a corresponding length of the allogeneic cartilage was cut out. The collected allogeneic cartilage of the corresponding length was implanted into the surface of the cartilage defect of the femoral head and fixed in the defect using absorbable screws. The surface of the costal cartilage was cut with a scalpel along the articular surface curvature of the femoral head to reshape the articular surface of the femoral head. Traction of the lower limbs, knee flexion, internal rotation to reset the hip joint were performed, moving the hip joint in all directions, so as to confirm that the costal cartilage was stable after implantation, and the costal cartilage was not in abnormal contact with the acetabular edge, such as impact. The range of passive motion of the hip joint was determined, and it was confirmed that the implanted cartilage was stable. The femoral head was good in shape through the observation of C-arm fluoroscopy. The joint capsule was sutured and was rinsed with a large amount of normal saline layer by layer and all of the incisions were closed. After the surgery, the patient was asked to exercise a large range of motion of the joints passively in full range, and was prohibited from performing adduction and extorsion motions of the hip joint. On the second day after the surgery the patient can walk by the aid of crutches. After two weeks from the surgery, the patient begins to carry out full-range of exercises actively, and to train on the muscle strength step by step. After three months from the surgery full weight-bearing walking is regained gradually.

### EXAMPLE 4

This example is a method for repairing a cartilage defect on a knee joint by implanting allogeneic costal cartilage comprising the following steps:

Collecting and storing the allogeneic cartilage: the costal cartilage from a young donor was collected and placed in a preservation solution (serum-free medium, pH buffered), and the preservation solution was removed during use;

The patient was placed in a supine position under general anesthesia. After sterilization and draping, exsanguination was performed on the affected limb to reduce bleeding. A medial parapatellar incision was made longitudinally in the left knee in superficial and deep fascia, an incision was made on the media patellar retinaculum, and the osteochondral defect site of the femoral condyle was exposed. The degenerative cartilage and subchondral bone were cleared until the edge of normal bleeding, and the edge trimmed such that the border was perpendicular to the bone bed. Iliac bone may be collected for implantation depending on the size of the bone defect. A corresponding length of the allogeneic cartilage was cut out and implanted into the surface of the cartilage defect. The implant was fixed using absorbable screws, and the surface of the costal cartilage was cut with the scalpel along the articular surface curvature of the femoral head to reshape the articular surface of the knee joint. Knee flexion-extension motions were performed, so as to confirm that the graft was stable and was fixed in the cartilage defect site, shape curvature was satisfactory, range of the flexion-extension motions were not limited, and there was no abnormal contact and friction. After a drainage tube was indwelled, the wound was closed by suture layer by layer. Postoperatively the patient began passive knee motion fir the first day. Mobilization on crutches was allowed without weight-hearing on the second day. At 4 weeks postoperatively, the knee motion was from 0° to 120°. After 12 weeks, weight bearing was progressed gradually as tolerated. When comparing this embodiment with the traditional allogeneic osteochondral graft, this embodiment of the present invention simply used the cartilage, which does not contain bone components. In this case, immune rejection was avoided to a certain extent and the rejection reaction was small. The secondary damage was smaller than autologous cartilage implantation.

### EXAMPLE 5

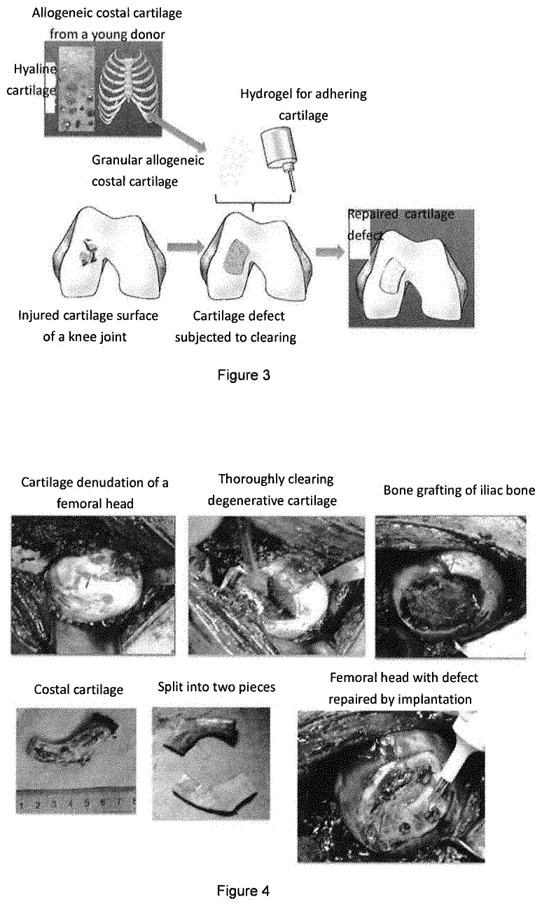

This embodiment is a method for repairing a cartilage defect on a knee joint by implanting allogeneic costal cartilage particles comprising the following steps, and as shown in FIG. 3:

Preparing minced hyaline cartilage pieces: costal cartilage was collected from a young donor and cut into particles of 0.1 mm to 1.5 mm;

The minced cartilage particles were mixed with a phototriggered-imine-croslinking hydrogel system, so as to form a graft, wherein the granular cartilages account for 30%-85% of the total weight of the system.

An osteochondral defect on a knee joint was repaired by filling the defect with the above-disclosed graft. The phototriggered-imine-croslinking hydrogel may be securely attached to the cartilage defect to form an integrated seamless surface and facilitating integration of new cartilage into surrounding cartilage tissues.

The method specifically comprises the following steps:

The patient was placed in a supine position under general anesthesia. After sterilization and draping, exsanguination was performed on the affected limb to reduce bleeding. A medial parapatellar incision was made longitudinally in the left knee in superficial and deep fascia, an incision was made on the media patellar retinaculum, and the osteochondral defect site of the femoral condyle was exposed. The degenerative cartilage and subchondral bone were cleared until the edge of normal bleeding and the edge trimmed such that the border is perpendicular to the bone bed.

Fresh allogeneic costal cartilage particles were pre-stored in a preservation solution. A syringe was used to remove as much preservation solution as possible. An appropriate amount of costal cartilage particles were mixed with hydrogel (1:1) according to the size of the defect, and the mixture of the costal cartilage particles and hydrogel was implanted into the cartilage defect, distributing cartilage particles evenly into the defect. The surface was shaped with a finger or a tool to the surface of the defect, such that the graft is consistent with the curvature of the articular surface and lower than the surrounding cartilage surface by about 0.5 mm. The excess liquid was removed, and the :filling portion was irradiated with an LED light source (20 mW/cm²) for 3 minutes to fully form a gel. It was confirmed again that the graft was stable, fixed in the cartilage defect site, and the shape curvature satisfactory. After a drainage tube was indwelled, the incision was closed suture layer by layer. Postoperatively, the patient began passive knee motion for the first day. Mobilization on crutches was allowed without weight-bearing on the second day. At 4 weeks postoperatively, the knee motion was from 0° to 120′. After 12 weeks, weight hearing was progressed gradually as tolerated. When comparing this embodiment with the traditional allogeneic osteochondral graft, the present invention simply used the cartilage, which does not contain bone components. In this case, immune rejection was avoided to a certain extent, and the rejection reaction was small. The secondary damage was smaller than autologous cartilage implantation.

### EXAMPLE 6

This embodiment is a corresponding clinical application. The patient was 24 years old having pain in his right hip, and claudication had lasted for more than 3 months. The diagnosis was as follows: “Femoral head cartilage injury caused by necrosis of the femoral head”. Artificial joint replacement was the only method to repair the femoral head cartilage injury caused by necrosis of the femoral head. However, the artificial joint had limited time of service life and, for young adults, about 20 years old, if an artificial joint replacement was performed, then repeated replacements may occur accordingly. In this case, complications such as prosthesis loosening and infection may be disastrous for the patient. Therefore, it was decided to repair and reconstruct the cartilage injury of the patient by using the costal cartilage graft. The repair and reconstruction process is shown below, and as shown in FIG. 4:

Under general anesthesia, the patient was placed in the supine position, incisions were made on skin, subcutaneous tissues, and fascia lata in sequence by using Smith-Peterson approach, but taking care to avoid damaging anterolateral femoral cutaneous nerves, to fully expose the anterior joint capsule of the hip joint, and incisions were made in a “T”-shape. After fully loosening the hip joint, the femur was at adduction and extorsion, and the femoral head was exposed. The degenerative necrotic bone and cartilage tissues of the femoral head were thoroughly cleared until fresh bleeding occurred. According to the severity of the bone defect, the iliac bone block on the same side in the same incision was collected to reconstruct the subchondral bone defect of the femoral head. While one group of doctors were performing the hip joint surgery, another group of doctors stood the right side to collect the costal cartilage, and to make an incision on the skin along a long axis of the right 6th rib (moving window technique), wherein the incision was about 6 cm long, but taking care to avoid damaging the intercostal vessels and nerves. According to the size of the femoral head osteochondral detect, costal cartilage of about 5 to 6 cm long was cut out with a scalpel. After confirming that pleura was intact without any injury, the rib periosteum was sutured, and the incision on the chest was closed. The iliac bone block was repaired and embedded into the subchondral bone defect in the weight-bearing area of the femoral head and trimmed to the shape. The costal cartilage was longitudinally split into two pieces with a scalpel, and then the two pieces of costal cartilage were implanted to the surface of the femoral head cartilage defect site. The costal cartilage and the iliac bone block were fixed with six absorbable screws and the surface of the costal cartilage was cut with the scalpel along the articular surface curvature of the femoral head to reshape the articular surface of the femoral head. Traction of the lower limbs, knee flexion, internal rotation to reset the hip joint were performed, moving the hip joint in all directions, so as to confirm that the costal cartilage was stable after implantation and not in abnormal contact with the acetabular edge, such as impact. Range of motion of the hip joint was determined: 0°-75° forward flexion, 0°-10° backward extension, 0°-40° abduction, 0°-25° adduction, 0°-30° extorsion and 0°-30° intorsion when the hip joint does forward flexion, and the implanted cartilage of the bone block was stable. The femoral head was good in shape through the observation of C-arm fluoroscopy. The joint capsule was sutured and was rinsed with a large amount of normal saline layer by layer and all of the incisions were closed. After the surgery, the patient was asked to exercise a large range of motion of the joints passively in all directions, and was prohibited from perthrming adduction and extorsion motions of the hip joint. On the second day after the surgery, the patient could walk by the aid of crutches. After two weeks from the surgery, the patient began to carry out full-range of exercises actively, and to train on muscle strength step by step. After three months from the surgery, full weight-bearing walking was regained gradually. The patient was followed up for 9 months, and it was found that the medical condition improved and that the injured hip became nearly normal.

From the above-mentioned examples, a small secondary injury can be treated by using costal cartilage implantation. In addition, it will not damage normal articular cartilage, and it can be a minimally invasive operation, avoiding the risk of performing premature artificial joint replacements on young patients and complications due to the artificial joint replacements. An effective method for repairing the cartilage injury of the femur head is herein provided. Because the amount of costal cartilage is sufficient, it can meet the needs of multiple cartilage reconstruction and revision surgeries. Individualized reconstruction of injured articular cartilage surface can be performed, which provides a safer, more operable repair method for patients diagnosed with articular cartilage defects.

The above descriptions are only the preferred embodiments of the invention, not thus limiting the embodiments and scope of the invention. Those skilled in the art should be able to realize that the schemes obtained from the content of specification and drawings of the invention are within the scope of the invention.

What is claimed is:

1. A graft for repairing articular cartilage defects, wherein the graft comprises at least one of an autologous costal cartilage or an allogeneic costal cartilage.

2. The graft for repairing articular cartilage defects as claimed in claim 1, wherein the costal cartilage does not have an osteochondral joint.

3. The graft for repairing articular cartilage defects as claimed in claim 1, wherein the costal cartilage comprises a whole piece of costal cartilage or granular cartilage.

4. The graft for repairing articular cartilage defects as claimed in claim 3, wherein the granular cartilage has a size between 0.1 mm to 1.5 mm.

5. The graft for repairing articular cartilage defects as claimed in claim 3, wherein the graft further comprises a hydrogel system.

6. The graft for repairing articular cartilage defects as claimed in claim 5, wherein the hydrogel system is selected from the group consisting of: autologous fibrin, autologous platelet-rich plasma, synthetic collagen, gelatin, chitosan, and polymer hydrogel.

7. Use of an autologous costal cartilage or an allogeneic costal cartilage in the preparation of a graft for repairing articular cartilage defects.

8. A method for repairing articular cartilage defects, the method comprising applying a graft to a defect site to be repaired, wherein the graft comprises at least one of an autologous costal cartilage or an allogeneic costal cartilage.

9. The method for repairing articular cartilage defects as claimed in claim 8, wherein the costal cartilage does not have an osteochondral joint.

10. The method for repairing articular cartilage defects as claimed in claim 8, wherein the costal cartilage is a whole piece of costal cartilage or granular cartilage.

11. The method for repairing articular cartilage defects as claimed in claim 10, wherein the granular cartilage has a size between 0.1 mm to 1.5 mm.

12. The method for repairing articular cartilage defects as claimed in claim 10, wherein the graft further comprises a hydrogel system.

13. The method for repairing articular cartilage defects as claimed in claim 12, wherein the hydrogel system is selected from the group consisting of autologous fibrin, autologous platelet-rich plasma, synthetic collagen, gelatin, chitosan, and polymer hydrogel.

14. The method for repairing articular cartilage defects as claimed in claim 8, wherein the graft comprises autologous costal cartilage and/or allogeneic costal cartilage, and the articular cartilage defect is cartilage defect on a femoral head or osteochondral defect on a knee joint, the method for repairing articular cartilage defects comprises the following steps:
(1) in an injured articular cartilage surface, clearing necrotic and degenerative cartilages and subchondral bone tissues, and preparing a viable bone bed for implantation of the costal cartilage;
(2) cutting out a corresponding length of the costal cartilage depending on a size of the cartilage defect for collecting the autologous costal cartilage or the allogeneic costal cartilage; and
(3) implanting the autologous costal cartilage or the allogeneic costal cartilage into the bone bed formed by the removal of the cartilage defect, and securing the autologous costal cartilage or the allogeneic costal cartilage in the bone bed and cutting the same into desired shapes, so as to atch the cartilage surface with a surrounding cartilage surface and articular surface morphology.

15. The method for repairing articular cartilage defects as claimed in claim 8, wherein the graft is autologous costal cartilage and/or allogeneic costal cartilage, the articular cartilage defect is the knee joint osteochondral injury, and the method for repairing articular cartilage defects comprises the following steps:
(A) collecting at least one of the autologous costal cartilage and the allogeneic costal cartilage, cutting the same into particles having a size of 0.1 mm to 1.5 mm, and mixing cartilage particles with the hydrogel system, so as to form a graft consisting of the cartilage particles and the hydrogel system;
(B) in an injured articular cartilage surface, clearing necrotic and degenerative cartilages and subchondral bone tissues, and preparing a bone bed to be processed; and
(C) repairing a knee joint osteochondral defect by :filling in the graft to form an integration seamless surface, and facilitating integration of new cartilages into surrounding cartilage tissues.
